(19) United States
(12) Patent Application Publication
(10) Pub. No.: US 2026/0077109 A1
(43) Pub. Date: Mar. 19, 2026

(54) Determination Of Red Blood Cell Recovery

(71) Applicant: Fenwal, Inc., Lake Zurich, IL (US)

(72) Inventors: Benjamin E. Kusters, Pleasant Prairie, WI (US); Kyungyoon Min, Kildeer, IL (US)

(21) Appl. No.: 19/250,540

(22) Filed: Dec. 8, 2025

Related U.S. Application Data
(60) Provisional application No. 63/666,340, filed on Jul. 1, 2024.

(51) Int. Cl. A61M 1/02; A61M 1/36

(52) U.S. Cl. CPC A61M 1/0272; A61M 1/0218; A61M 1/3609; A61M 2202/0014; A61M 2202/0429; A61M 2205/103; A61M 2205/12; A61M 2205/3306; A61M 2205/3334; A61M 2205/3379; A61M 2205/50; A61M 2205/7545

(57) ABSTRACT

A blood separation device includes a pump system, a separator, and a controller. The controller is programmed to execute a blood separation procedure including a red blood cell collection stage in which blood is conveyed into the separator, red blood cells are separated from the blood in the separator, and the separated red blood cells are conveyed from the separator into a collection container. The red blood cell collection stage is ended when a target volume of blood has been separated in the separator. The controller then determines a percentage of the red blood cells from the target volume of blood that have been collected in the collection container based on the volume of red blood cells and the hematocrit of the fluid in the collection container, a volumetric flow rate of pure red blood cells in the blood conveyed into the separator, and the procedure time.

### CROSS-REFERENCE TO RELATED APPLICATIONS

This application claims the benefit of and priority of U.S. Provisional Patent Application Ser. No. 63/666,340, filed Jul. 1, 2024, the contents of which are incorporated by reference herein.

### BACKGROUND

### Field of the Disclosure

The present disclosure relates to separation of red blood cells from blood. More particularly, the present disclosure relates to determination of the percentage of red blood cells from a volume of whole blood that is separated from the whole blood and subsequently recovered in a collection container.

### Description of Related Art

Various blood processing systems now make it possible to collect particular blood constituents, instead of whole blood, from a blood source. Typically, in such systems, whole blood is drawn from a blood source, the particular blood component or constituent is separated, removed, and collected, and the remaining blood constituents are returned to the blood source. Removing only particular constituents is advantageous when the blood source is a human donor, because potentially less time is needed for the donor's body to return to pre-donation levels, and donations can be made at more frequent intervals than when whole blood is collected. This increases the overall supply of blood constituents, such as plasma and platelets, made available for transfer and/or therapeutic treatment.

According to one approach, whole blood may be separated into its constituents through centrifugation. This requires that the whole blood be passed through a centrifuge after it is withdrawn from, and before it is returned to, the blood source. To reduce contamination and possible infection (if the blood source is a human donor or patient), the blood is preferably processed within a sealed, sterile fluid flow circuit during the centrifugation process. The operator installs afresh, sterile disposable flow circuit in the centrifuge before processing and removes and discards it afterwards. Typical disposable flow circuits are sealed and sterile, and include a separation chamber portion, which is mounted in cooperation on a durable, reusable assembly containing the hardware (centrifuge, drive system, pumps, valve actuators, programmable controller, and the like) that rotates the separation chamber and controls the flow through the fluid circuit. The separation chamber may be formed of a generally rigid material (e.g., molded plastic), in which case the chamber itself defines a flow path or channel in which blood is separated into two or more components, or a more flexible material (e.g., in the form of a belt or annulus), which relies upon the system hardware to support the chamber and define the shape of the chamber as blood flows through it.

With a disposable circuit loaded onto the centrifuge (or just prior to or during loading) the operator typically enters, for example, by means of a touch screen or other user interface system, a particular processing protocol to be executed by the system (e.g., a procedure wherein platelets are separated from whole blood and collected) and other parameters (e.g., the weight of the donor, the desired volume of separated blood component to be collected, etc.). When the system has been programmed, the operator phlebotomizes a donor (or otherwise accesses the blood of the blood source) and the system carries out the procedure, under the supervision of the operator.

The centrifuge rotates the separation chamber of the disposable flow circuit during processing, causing the heavier (greater specific gravity) components of the whole blood in the separation chamber, such as red blood cells, to move radially outwardly away from the center of rotation toward the outer or “high-G” wall of the separation chamber. The lighter (lower specific gravity) components, such as plasma, migrate toward the inner or “low-G” wall of the separation chamber. The boundary that forms between the heavier and lighter components in the separation chamber is commonly referred to as the interface. Various ones of these components can be selectively removed from the whole blood by providing appropriately located channeling structures and outlet ports in the flow circuit. For example, in one blood separation procedure, blood is separated into plasma and red blood cells, with a buffy coat or white blood cell layer positioned therebetween. The buffy coat or white blood cell layer (which may include platelets and various white blood cells, such as mononuclear cells and possibly peripheral blood stem cells) is collected, with plasma and red blood cells being returned to the blood source or separately collected. A system capable of executing such a red blood cell, plasma, and buffy coat collection procedure is described in PCT Patent Application Publication No. WO 2021/194824 A1, which is hereby incorporated herein by reference. In addition to centrifugation, other approaches to separation of blood into its constituents are known and widely practiced, including separation of blood using a spinning membrane-type separator, with an exemplary spinning membrane-type separator being described in U.S. Patent Application Publication No. 2019/0201916, which is hereby incorporated herein by reference.

In the case of procedures in which red blood cells are separated from blood and collected, conventional approaches for determining the red blood cell recovery during the procedure focus on the recovery of red blood cells passed through a leukoreduction filter, applying a ratio of the red blood cell content (determined from weight and hematocrit) of a red blood cell product pre- and post-filtration to determine the recovery of red blood cells through the filter. These methods rely on use of quality controls and testing to ensure that a certain percentage of leukoreduction processes will meet red blood cell recovery standards, with one approach operating based on the understanding or presumption that 85% of red blood cells that pass into a filter will subsequently pass into the downstream red blood cell product bag 95% of the time. It will, thus, be seen that, in these conventional approaches, the actual red blood cell recovery of any single iteration of a procedure is unknown, considering that the hematocrits of the products are not known prior to processing of the individual units during quality testing.

It would, thus, be advantageous to provide an approach by which the red blood cell recovery of a particular iteration of a blood separation procedure can be determined.

### SUMMARY

There are several aspects of the present subject matter which may be embodied separately or together in the devices and methods described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a blood separation device includes a pump system, a separator, and a controller operably connected to the pump system and the separator. The controller is programmed to execute blood separation procedure including a red blood cell collection stage in which the controller actuates the pump system to convey blood into the separator, actuates the separator to separate red blood cells from the blood in the separator, and actuates the pump system to convey the separated red blood cells from the separator into a collection container. The controller ends the red blood cell collection stage when a target volume of blood has been separated in the separator. The controller is further programmed to determine a percentage of the red blood cells from the target volume of blood that have been collected in the collection container using the equations:

$$\% R B C Recovery = \frac{Total Volume R B C s in R B C Bag}{Total Volume R B C s in WB Bag} \times 100 ,$$

Total Volume RBCs in RBC Bag=V_{RBC Bag}*H_{RBC Bag}, and
Total Volume RBCs in WB Bag=Q_{pureRBC-WB}*Procedure Time. In the equations, Total Volume RBCs in RBC Bag is a volume of red blood cells in the collection container at the end of the blood separation procedure, Total Volume RBCs in WB Bag is the target volume of blood, V_{RBC Bag} is a volume of fluid in the collection container at the end of the blood separation procedure, H_{RBC Bag} is a hematocrit of the fluid in the collection container at the end of the blood separation procedure, Q_{pureRBC-WB} is a volumetric flow rate of pure red blood cells in the blood conveyed into the separator during the red blood cell collection stage, and Procedure Time is the amount of time during the red blood cell collection stage when red blood cells are being separated from the blood in the separator.

In another aspect, a controller-implemented method of executing a blood separation procedure is provided. The method includes executing a red blood cell collection stage in which blood is conveyed into a separator, red blood cells are separated from the blood in the separator, and the separated red blood cells are conveyed from the separator into a collection container. The red blood cell collection stage is ended when a target volume of blood has been separated in the separator. The controller then determines a percentage of the red blood cells from the target volume of blood that have been collected in the collection container using the equations:

$$\% R B C Recovery = \frac{Total Volume R B C s in R B C Bag}{Total Volume R B C s in WB Bag} \times 100 ,$$

Total Volume RBCs in RBC Bag=V_{RBC Bag}*H_{RBC Bag}, and Total Volume RBCs in WB Bag=Q_{pureRBC-WB}*Procedure Time. In the equations, Total Volume RBCs in RBC Bag is a volume of red blood cells in the collection container at the end of the blood separation procedure, Total Volume RBCs in WB Bag is the target volume of blood, V_{RBC Bag} is a volume of fluid in the collection container at the end of the blood separation procedure, H_{RBC Bag} is a hematocrit of the fluid in the collection container at the end of the blood separation procedure, Q_{pureRBC-WB} is a volumetric flow rate of pure red blood cells in the blood conveyed into the separator during the red blood cell collection stage, and Procedure Time is the amount of time during the red blood cell collection stage when red blood cells are being separated from the blood in the separator.

### BRIEF DESCRIPTION OF THE DRAWINGS

FIG. 1 is a perspective view of an exemplary reusable hardware component of a blood separation system which is configured to receive a disposable fluid flow circuit;

FIG. 2 is a plan view of an exemplary disposable fluid flow circuit for use in combination with the durable hardware component of FIG. 1;

FIG. 3 is a schematic view of the fluid flow circuit of FIG. 2 mounted to the blood separation device of FIG. 1 to complete a blood separation system according to an aspect of the present disclosure; and

FIG. 4 is a schematic view of the exemplary blood separation system of FIG. 3 executing a “red blood cell collection” stage of an exemplary blood separation procedure.

### DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific designs and features disclosed herein are not to be interpreted as limiting the subject matter as defined in the accompanying claims.

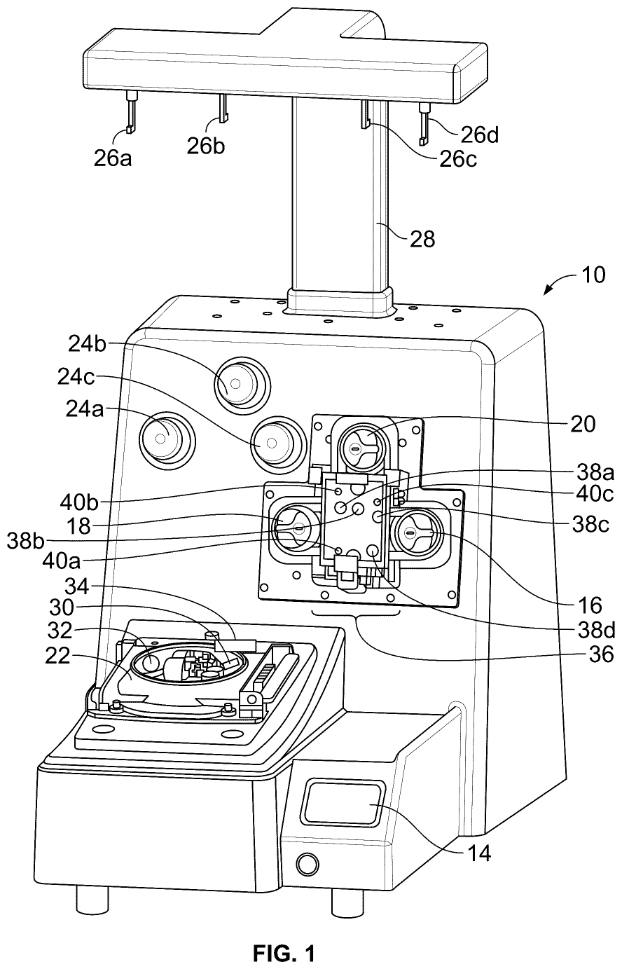

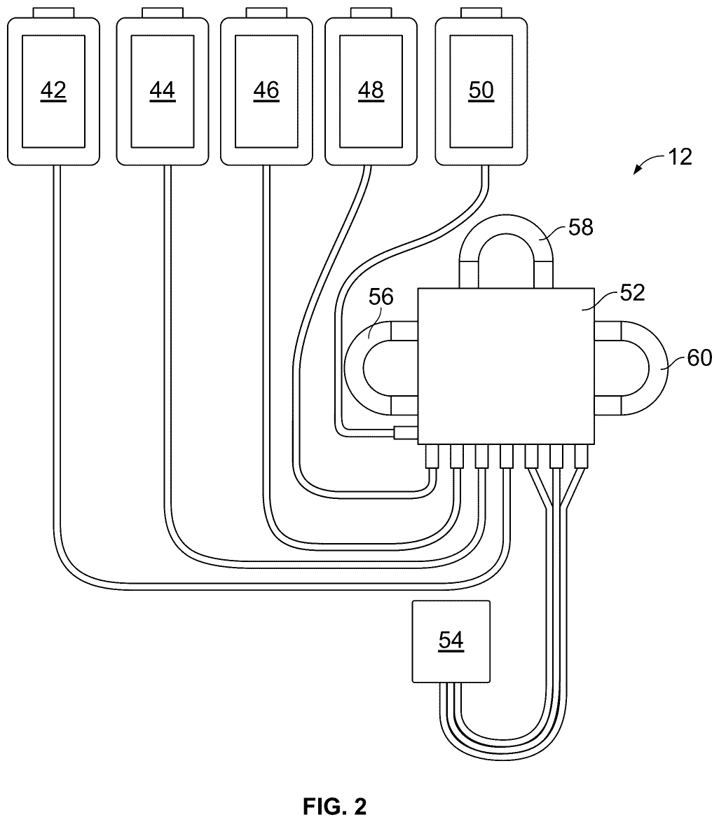

FIG. 1 depicts a reusable hardware component or separation device of a blood separation system, generally designated 10, while FIG. 2 depicts a disposable fluid flow circuit, generally designated 12, to be used in combination with the separation device 10 for separating blood into two or more components. The separation device 10 and fluid flow circuit 12 are configured in general accordance with the description provided in PCT Patent Application Publication No. WO 2021/194824 A1, except where noted herein to the contrary. It should be understood that, while the principles described herein may be particularly advantageous for a separation device of the type shown in FIG. 1, these principles are applicable to differently configured blood separation devices and systems, including those separation devices employing a separator that is not configured as a centrifuge.

The illustrated separation device 10 includes associated pumps, valves, sensors, displays, and other apparatus for configuring and controlling flow of fluid through the fluid flow circuit 12, as will be described in greater detail below. The fluid separation system may be directed by a controller integral with the separation device 10 that automatically controls the operation of the pumps, valves, sensors, etc. The controller may be variously configured without departing from the scope of the present disclosure. In one embodiment, the controller may include a microprocessor (which, in fact may include multiple physical and/or virtual processors). According to other embodiments, the controller may include one or more electrical circuits designed to carry out the actions described herein. In fact, the controller may include a microprocessor and other circuits or circuitry. In addition, the controller may include one or more memories. The instructions by which the microprocessor is programmed may be stored on the memory associated with the microprocessor, which memory/memories may include one or more tangible non-transitory computer readable memories, having computer executable instructions stored thereon, which when executed by the microprocessor, may cause the microprocessor to carry out one or more actions as described herein. In one exemplary embodiment, the controller comprises a main processing unit (MPU), which can comprise, e.g., a PENTIUM® type microprocessor made by Intel Corporation, although other types of conventional microprocessors can be used. In addition to the controller, the separation device 10 may also include wireless communication capabilities to enable the transfer of data from the separation device 10 to the quality management systems of the operator.

More specifically, the illustrated separation device 10 includes a user input and output touchscreen 14, a pump station including a first pump 16 (for pumping, e.g., whole blood), a second pump 18 (for pumping, e.g., plasma) and a third pump 20 (for pumping, e.g., additive solution), a separator or centrifuge mounting station and drive unit 22 (which may be referred to herein as a “centrifuge”), and clamps 24a-c. The touchscreen 14 enables user interaction with the separation device 10, as well as the monitoring of procedure parameters, such as flow rates, container weights, pressures, etc. The pumps 16, 18, and 20 (collectively referred to herein as being part of a “pump system” of the separation device 10) are illustrated as peristaltic pumps capable of receiving tubing or conduits and moving fluid at various rates through the associated conduit dependent upon the procedure being performed. The clamps 24a-c (collectively referred to herein as being part of the “valve system” of the separation device 10) are capable of opening and closing fluid paths through the tubing or conduits and may incorporate RF sealers in order to complete a heat seal of the tubing or conduit placed in the clamp to seal the tubing or conduit leading to a product container upon completion of a procedure.

The separation device 10 also includes hangers 26a-d (which may each be associated with a weight scale) for suspending the various containers of the disposable fluid circuit 12. The hangers 26a-d are preferably mounted to a support 28, which is vertically translatable to improve the transportability of the separation device 10. An optical system comprising a laser 30 and a photodetector 32 is associated with the centrifuge 22 for determining and controlling the location of an interface between separated blood components within the centrifuge 22. An exemplary optical system is shown in U.S. Patent Application Publication No. 2019/0201916. An optical sensor 34 is also provided to optically monitor one or more conduits leading into or out of the centrifuge 22.

The face of the separation device 10 includes a nesting module 36 for seating a flow control cassette 52 (FIG. 2) of the fluid flow circuit 12. The cassette nesting module 36 is configured to receive various disposable cassette designs so that the system may be used to perform different types of procedures. Embedded within the illustrated cassette nesting module 36 are four valves 38a-d (collectively referred to herein as being part of the “valve system” of the separation device 10) for opening and closing fluid flow paths within the flow control cassette 52, and three pressure sensors 40a-c capable of measuring the pressure at various locations of the fluid flow circuit 12.

With reference to FIG. 2, the illustrated fluid flow circuit 12 includes a plurality of containers 42, 44, 46, 48, and 50 with a flow control cassette 52 and a separation chamber 54 that is configured to be received in the centrifuge 22, all of which are interconnected by conduits or tubing segments, so as to permit continuous flow centrifugation. The flow control cassette 52 routes the fluid flow through three tubing loops 56, 58, 60, with each loop being positioned to engage a particular one of the pumps 16, 18, 20. The conduits or tubing may extend through the cassette 52, or the cassette 52 may have pre-formed fluid flow paths that direct the fluid flow.

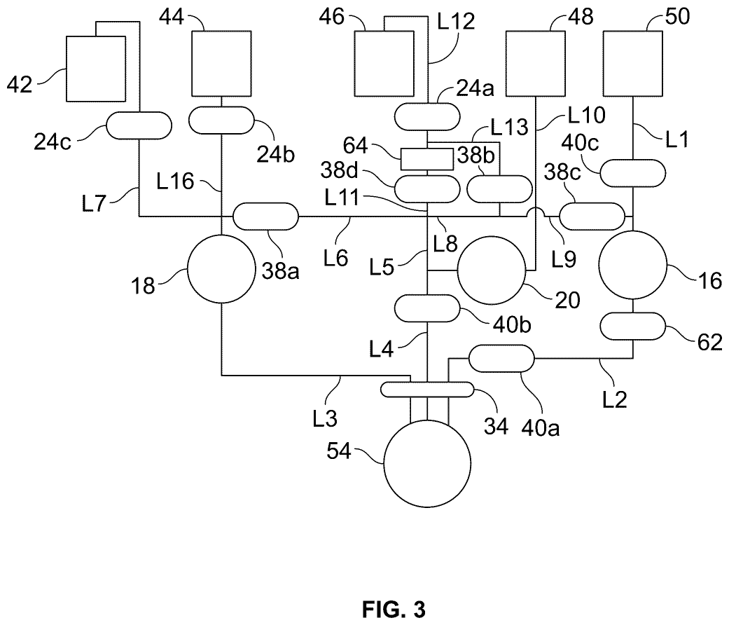

In the fluid flow circuit 12 shown in FIG. 2, containers 42, 44, and 46 may be empty containers for the receipt of plasma, a buffy coat or white blood cell layer, and red blood cells (respectively) separated from whole blood, container 48 may be pre-filled with additive solution, and container 50 may be filled with whole blood and connected to the fluid flow circuit 12 at the time of use. While FIG. 2 shows a whole blood container 50 (configured as a blood pack unit, for example) as a fluid source, it is within the scope of the present disclosure for the fluid source to be a living donor. The fluid flow circuit 12 may optionally include an air trap 62 (FIG. 3) through which the fluid to be separated is flowed prior to entering the separation chamber 54 and/or a filter 64 through which a separated fluid component is flowed prior to entering an associated container.

The separation chamber 54 may be pre-formed in a desired shape and configuration by injection molding from a rigid plastic material, as shown and described in U.S. Pat. No. 6,849,039, which is hereby incorporated herein by reference. The specific geometry of the separation chamber 54 may vary depending on the elements to be separated, and the present disclosure is not limited to the use of any specific chamber design. For example, it is within the scope of the present disclosure for the separation chamber 54 to be configured formed of a generally flexible material, rather than a generally rigid material. When the separation chamber 54 is formed of a generally flexible material, it relies upon the centrifuge 22 to define a shape of the separation chamber 54. An exemplary separation chamber formed of a flexible material and an associated centrifuge are described in U.S. Pat. No. 6,899,666, which is hereby incorporated herein by reference.

The controller of the separation device 10 may be pre-programmed to automatically operate the system to perform one or more standard fluid separation procedures selected by an operator by input to the touchscreen 14, and configured to be further programmed by the operator to perform additional procedures. The controller may be pre-programmed to substantially automate a wide variety of procedures, including, but not limited to: red blood cell and plasma production from a single unit of whole blood, buffy coat pooling, buffy coat separation into a platelet product, glycerol addition to red blood cells, red blood cell washing, platelet washing, and cryoprecipitate pooling and separation.

The pre-programmed separation procedures operate the system at pre-set settings for flow rates and centrifugation forces, and the programmable controller may be further configured to receive input from the operator as to one or more of flow rates and centrifugation forces for the standard separation procedure to override the pre-programmed settings. In addition, the programmable controller may be configured to receive input from the operator through the touchscreen 14 for operating the system to perform a non-standard separation procedure.

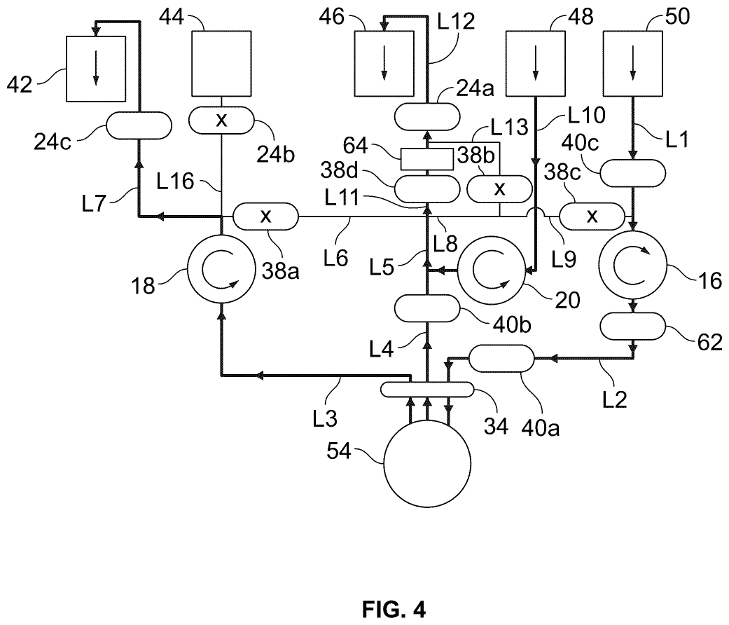

According to an aspect of the present disclosure, the separation device 10 and the fluid flow circuit 12 may be used in combination to execute a procedure having a “red blood cell collection” stage (FIG. 4). Any other stages may be executed before or after the “red blood cell collection” stage without departing from the scope of the present disclosure. For example, it may be typical for a blood separation procedure to begin with a “priming” stage in which a priming fluid is pumped through the fluid flow circuit 12 to move air into a designated location before beginning blood separation. It may also be common for a procedure to include an “additive solution flush” stage after red blood cells have been collected in order to ensure that the proper amount of an additive solution has been added to the collected red blood cells.

Regardless of the exact stages that are executed during a blood separation procedure, in the illustrated “red blood cell collection” stage, valve 38c is closed and pump 16 (which may be referred to as the “whole blood” pump) is operated to draw blood into line L1 from the blood source (which is the whole blood container 44 in the illustrated embodiment, but may be a living donor). The whole blood pump 16 draws the blood from the blood source into line L2 from line L1, with the blood passing through air trap 60, pressure sensor 40a, and optical sensor 34 before flowing into the separation chamber 54, where it is separated into plasma and red blood cells. Depending on the rate at which the centrifuge 22 is rotated (which may be in a range of approximately 4,500 to 5,500 rpm, for example), most of the platelets of the whole blood will remain in the separation chamber 54, along with some white blood cell populations (much as mononuclear cells), while larger white blood cells, such as granulocytes, may exit with the packed red blood cells.

The separated plasma exits the separation chamber 54 via a plasma outlet port of the separation chamber 54 and associated line L3. Valve 38a and clamp 24b are closed, which directs the plasma from line L3 into line L7, through open clamp 24c, and into the plasma collection container 48. The separated red blood cells exit the separation chamber 54 via a red blood cell outlet port of the separation chamber 54 and associated line L4. It will be seen that no pump is associated with line L4, such that the rate at which the red blood cells exit the separation chamber 54 will be equal to the difference between the rate at which the whole blood is pumped into the separation chamber 54 (the whole blood inflow rate) and the rate at which the plasma is pumped out of the separation chamber 54 (the plasma outflow rate). In other embodiments, a dedicated pump may be associated with line L4 to directly control the rate at which the separated red blood cells are removed from the separation chamber 54.

In the illustrated embodiment, pump 20 (which may be referred to as the “additive” pump) is operated by the controller to draw an additive solution (which is ADSOL® in one exemplary embodiment, but may be some other red blood cell additive) from the additive solution container 48 via line L10. The red blood cells flowing through line L4 are mixed with the additive solution flowing through line L10 at a junction of the two lines L4 and L10 to form a mixture that continues flowing into and through line L5. The mixture is ultimately directed into the red blood cell collection container 46, but may first be conveyed through a leukoreduction filter 64 (if provided), as shown in FIG. 4. Even if a leukoreduction filter 64 is provided, the valve system may be controlled to cause the mixture to bypass the leukoreduction filter 64 and enter the red blood cell collection container 46 without being leukoreduced. It is also within the scope of the present disclosure for the mixture to be routed through the leukoreduction filter 64 at the beginning of the “red blood cell collection” stage, with the valve system being reconfigured during the stage to cause the mixture to bypass the leukoreduction filter 64, such that only a portion of the collected red blood cells are leukoreduced.

In the configuration of FIG. 4 (in which the mixture is leukoreduced), valves 38a, 38b, and 38c are closed, while valve 38d is open, which directs the mixture from line L5 into line L11. The mixture flows through open valve 38d and the leukoreduction filter 64 and into line L12. The leukoreduced mixture then flows through open clamp 24a and into the red blood cell collection container 46. In an alternative arrangement (in which the mixture is not leukoreduced), valves 38a, 38c, and 38d are closed, while valve 38b is open, which directs the mixture from line L5 into line L8 and then into line L13. The mixture flows through open valve 38b and into line L12, bypassing the leukoreduction filter 64. The non-leukoreduced mixture then flows through open clamp 24a and into the red blood cell collection container 46. As described above, the mixture may be routed through the leukoreduction filter 64 at the beginning of the collection stage (as in FIG. 4), with the valve system being reconfigured during the collection stage to cause the mixture to bypass the leukoreduction filter 64, such that only a portion of the collected red blood cells are leukoreduced. In one embodiment, when pressure sensor 40b detects that the pressure of the leukoreduction filter 64 has risen above a predetermined pressure threshold (which may be indicative of filter blockage), the controller may reconfigure the valve system to cause the mixture to bypass the leukoreduction filter 64. The system may then alert the operator that the red blood cell product was not leukoreduced.

Regardless of whether the collected red blood cells have been leukoreduced (or only partially leukoreduced), the collection stage continues until a target volume of blood (e.g., one unit) has been drawn into the fluid flow circuit 12 from the blood source and separated. In the case of a whole blood container 50 being used as a blood source (as in the illustrated embodiment) the collection stage may end when the whole blood container 50 (which is initially provided with the target volume of whole blood) is empty, with different approaches possibly being employed to determine when the whole blood container 50 is empty. For example, in one embodiment, pressure sensor 40c monitors the hydrostatic pressure of the whole blood container 50. An empty whole blood container 50 may be detected when the hydrostatic pressure measured by pressure sensor 40c is at or below a threshold value. Alternatively (or additionally), the weight of the whole blood container 50 may be monitored by a weight scale, with an empty whole blood container 50 being detected when the weight is at or below a threshold value. In the case of a living donor (or in the event that the whole blood container 50 is provided with more than the target volume of blood), the volumetric flow rate of the whole blood pump 16 may be used to determine when the target volume of whole blood has been drawn into the fluid flow circuit 12. Once the target volume of whole blood has been drawn into the fluid flow circuit 12 and separated, the controller will transition the procedure to the next stage of the procedure, which may vary without departing from the scope of the present disclosure.

Turning now to the manner in which red blood cell recovery of an individual iteration of a blood separation procedure can be determined, the percentage of the volume of red blood cells present in the red blood cell collection container 46 at the end of the procedure compared to the volume of red blood cells that were present in the volume of blood that was separated during the procedure (i.e., the target volume of blood) can be calculated using the following equation:

$$\% R B C Recovery = \frac{Total Volume R B C s in R B C Bag}{Total Volume R B C s in WB Bag} \times 100 , \qquad [ 1 ]$$

where
Total Volume RBCs in RBC Bag is the volume of red blood cells in the red blood cell collection container 46 at the end of the procedure, and
Total Volume RBCs in WB Bag is the volume of red blood cells that were present in the volume of blood that was separated during the procedure (i.e., the target volume of blood). It is again noted that this approach to determination of red blood cell recovery is not limited to procedures in which the entire contents of a blood container or bag are separated into red blood cells and other blood components, but encompasses procedures in which only a portion of the blood in a source container or bag is separated (e.g., when one unit of blood is processed from a bag containing more than one unit of blood). Additionally, as also noted above, this approach to determination of red blood cell recovery is not limited to separation of blood from a source container or bag, but encompasses procedures in which blood from a living source is separated.

Total Volume RBCs in RBC Bag may be determined using the following equation:

$$Total Volume R B C s in R B C Bag = {V}_{RBC Bag} * {H}_{RBC Bag} , \qquad [ 2 ]$$

where
V_{RBC Bag} is the volume of fluid in the red blood cell collection container 46 at the end of the procedure, and
H_{RBC Bag} is the hematocrit of the fluid in the red blood cell collection container 46 at the end of the procedure. These two values may be determined according to any suitable approach, which may include use of a weight scale associated with the red blood cell collection container 46 and/or monitoring of the volumetric flow rates at which fluids are conveyed into and out of the red blood cell collection container 46 during the procedure to determine V_{RBC Bag}.

As shown in FIG. 4 and described above, an additive solution may be conveyed into the red blood cell collection container 46 during the red blood cell collection stage of a procedure. As also described above, an additional volume of additive solution may be conveyed into the red blood cell collection container 46 after the red blood cell collection stage has ended in order to ensure that the proper volume of additive solution is in the red blood cell collection container 46 at the end of the procedure. Any additive solution remaining in the red blood cell collection container 46 at the end of the procedure will contribute to both of the V_{RBC Bag} and H_{RBC Bag} values. The ratio of red blood cells to additive solution in the red blood cell collection container 46 at the end of the procedure may vary without departing from the scope of the present disclosure, though it may be advantageous for the volume of additive solution conveyed into the red blood cell collection container 46 by the end of the procedure to be informed by the volume of red blood cells that has been collected. For example, in one embodiment, additive solution may be added to the red blood cells in the red blood cell collection container 46 until a target hematocrit has been achieved for the resulting fluid in the red blood cell collection container 46. In such an embodiment, the target hematocrit for the fluid in the red blood cell collection container 46 will be equal to H_{RBC Bag}.

Total Volume RBCs in WB Bag may be determined using the following equation:

$$Total Volume R B C s in WB Bag = {Q}_{pureRBC - WB} * Procedure Time , \qquad [ 3 ]$$

where
Q_{pureRBC-WB} is a volumetric flow rate of pure red blood cells in the blood conveyed into the separation chamber 54 during the red blood cell collection stage, and
Procedure Time is the amount of time during the red blood cell collection stage when red blood cells are being separated from the blood in the separation chamber 54. These two values may be determined according to any suitable approach without departing from the scope of the present disclosure.

In one embodiment, the determination of Q_{pureRBC-WB} includes the controller carrying out the blood separation procedure in a way that establishes steady state separation of the red blood cells from the blood in the separation chamber 54 before the pump system is actuated to convey the separated red blood cells from the separation chamber 54 into the red blood cell collection container 46 during the red blood cell collection stage. As used herein, the phrase “steady state separation” refers to a state in which blood is separated into its constituents in the separation chamber 54, with the radial position of the interface between separated components within the separation chamber 54 being at least substantially maintained (rather than moving radially inwardly or outwardly). The position of the interface may be determined and controlled according to any suitable approach, including using an optical system of the type described in U.S. Patent Application Publication No. 2019/0201916. Preferably, steady state separation is achieved with the interface between separated components within the separation chamber 54 at a target location, which may correspond to the location of the interface at which separation efficiency is optimized, with the precise location varying depending on a number of factors (e.g., the hematocrit of the whole blood). An exemplary approach to establishing steady state separation before fluid component collection is described in PCT Patent Application Publication No. WO 2021/194824 A1.

Establishing steady state separation before beginning red blood cell collection ensures that all of the red blood cells that enter the separation chamber 54 will exit the separation chamber 54, with no build up or loss of red blood cells in the separation chamber 54. When the separated red blood cells are collected under these conditions, the following equation is applicable:

$${Q}_{pureRBC - WB} = {Q}_{pureRBC - RBC} , \qquad [ 4 ]$$

where
Q_{pureRBC-RBC} is a volumetric flow rate of pure red blood cells in the separated red blood cells conveyed out of the separation chamber 54 during the red blood cell collection stage, which may be calculated according to the following equation:

$${Q}_{pureRBC - RBC} = {Q}_{RBC} * {H}_{RBC} , \qquad [ 5 ]$$

where
Q_{RBC} is a volumetric flow rate of the separated red blood cells conveyed out of the separation chamber 54 for collection during the red blood cell collection stage, and
H_{RBC} is the hematocrit of the separated red blood cells conveyed out of the separation chamber 54 for collection during the red blood cell collection stage. These two values may be determined according to any suitable approach without departing from the scope of the present disclosure.

As shown in FIG. 4 and described above, it may be the case that there is no pump associated with the line L4 by which the separated red blood cells are conveyed out of the separation chamber 54. In such an embodiment, Q_{RBC} will be equal to the difference between the rate at which the whole blood is pumped into the separation chamber 54 (the whole blood inflow rate) and the rate at which the plasma is pumped out of the separation chamber 54 (the plasma outflow rate). In other embodiments, a dedicated pump may be associated with line L4 to directly control the rate at which the separated red blood cells are removed from the separation chamber 54 for collection, in which case Q_{RBC} will be equal to the rate at which such pump operates while conveying the separated red blood cells from the separation chamber 54 into the red blood cell collection container 46.

As for H_{RBC}, one exemplary approach to determining its value is to set the value to a predetermined value based on the separation efficiency of the separator. For example, in one embodiment, it was empirically determined that the separation efficiency of a particularly configured centrifuge results in the separated red blood cells exiting the centrifuge having a hematocrit of approximately 80%. According to another approach, the optical sensor 34 may be employed to monitor line L4 (or a separate optical sensor may be employed to monitor line L12), with the optical sensor sending signals to the controller and the controller interpreting the signals in order to determine the hematocrit of the separated red blood cells exiting the separator. According to yet another approach, when a centrifuge is used to separate the red blood cells from blood, the controller may calculate H_{RBC} using a centrifugation-based separation efficiency equation, such as the following:

$${{H}_{RBC} ( 1 - {H}_{RBC} )}^{k + 1} = \frac{β {Q}_{i} {H}_{i}}{\frac{k + 2}{α} {( \frac{k + 2}{k + 1} )}^{k + 1} g {A}_{Sep} {S}_{R}} , \qquad [ 6 ]$$

where
α and k are different viscosity constants,
β is a strain rate-dependent shear stress factor,
Qᵢ is the flow rate at which the blood enters the centrifuge,
Hᵢ is the hematocrit of the blood,
g is centrifugal acceleration,
A_{Sep} is the separation chamber area, and
S_{R} is a red blood cell sedimentation coefficient.

Once the red blood cell recovery percentage has been determined by the controller, that information may be stored locally and/or presented to an operator and/or transferred to a facility data management system for storage and association to the red blood cell product and/or put to any other use. In one embodiment, the red blood cell recovery percentage may be used to identify insufficient red blood cell products, such as those with less than a predetermined total volume of red blood cells due to a poor recovery of the process, which may occur due to cell types (e.g., sickle cell), clots, or other system-related issues.

It should be understood that the illustrated separation device 10, the fluid flow circuit 12, and the above-described “red blood cell collection” stage are merely exemplary of a system and procedure that may used in combination with the red blood cell recovery calculation techniques and principles described herein. Indeed, differently configured blood separation systems and separation procedures including different stages may employ the techniques and principles described herein without departing from the scope of the present disclosure. This may include blood separation devices that do not rely upon centrifugation to separate red blood cells from blood, but employ a different approach to blood separation (e.g., blood separation via use of a spinning membrane-type separator).

### Aspects

Aspect 1. A blood separation device, comprising: a pump system; a separator; and a controller operably connected to the pump system and the separator and programmed to execute blood separation procedure including a red blood cell collection stage in which the controller actuates the pump system to convey blood into the separator, actuates the separator to separate red blood cells from the blood in the separator, actuates the pump system to convey the separated red blood cells from the separator into a collection container, and ends the red blood cell collection stage when a target volume of blood has been separated in the separator, wherein the controller is further programmed to determine a percentage of the red blood cells from the target volume of blood that have been collected in the collection container using the equations:

$$\% R B C Recovery = \frac{Total Volume R B C s in R B C Bag}{Total Volume R B C s in WB Bag} \times 100 ,$$

Total Volume RBCs in RBC Bag=V_{RBC Bag}*H_{RBC Bag}, and Total Volume RBCs in WB Bag=Q_{pureRBC-WB}*Procedure Time, wherein Total Volume RBCs in RBC Bag is a volume of red blood cells in the collection container at the end of the blood separation procedure, Total Volume RBCs in WB Bag is the target volume of blood, V_{RBC Bag} is a volume of fluid in the collection container at the end of the blood separation procedure, H_{RBC Bag} is a hematocrit of the fluid in the collection container at the end of the blood separation procedure, Q_{pureRBC-WB} is a volumetric flow rate of pure red blood cells in the blood conveyed into the separator during the red blood cell collection stage, and Procedure Time is the amount of time during the red blood cell collection stage when red blood cells are being separated from the blood in the separator.

Aspect 2. The blood separation device of Aspect 1, wherein the controller is further programmed to establish steady state separation of the red blood cells from the blood in the separator before actuating the pump system to convey the separated red blood cells from the separator into the collection container during the red blood cell collection stage.

Aspect 3. The blood separation device of Aspect 2, wherein the controller is further programmed to use the following equations to determine the percentage of the red blood cells from the target volume of blood that have been collected in the collection container: Q_{pureRBC-WB}=Q_{pureRBC-RBC}, and Q_{pureRBC-RBC}=Q_{RBC}*H_{RBC}, wherein Q_{pureRBC-RBC} is a volumetric flow rate of pure red blood cells in the separated red blood cells conveyed out of the separator during the red blood cell collection stage, Q_{RBC} is a volumetric flow rate of the separated red blood cells conveyed out of the separator during the red blood cell collection stage, and H_{RBC} is a hematocrit of the separated red blood cells conveyed out of the separator during the red blood cell collection stage.

Aspect 4. The blood separation device of Aspect 3, wherein the controller is further programmed to determine H_{RBC} to be equal to a predetermined value based on a separation efficiency of the separator.

Aspect 5. The blood separation device of Aspect 3, wherein the separator is configured as a centrifuge, and the controller is further programmed to calculate H_{RBC} using a centrifugation-based separation efficiency equation.

Aspect 6. The blood separation device of Aspect 3, further comprising an optical sensor configured to monitor the separated red blood cells conveyed out of the separator during the red blood cell collection stage, wherein the controller is programmed to receive signals from the optical sensor and to determine H_{RBC} based at least in part on said signals.

Aspect 7. The blood separation device of any one of the preceding Aspects, wherein the controller is further programmed to actuate the pump system to convey an additive solution into the collection container during the red blood cell collection stage.

Aspect 8. The blood separation device of Aspect 7, wherein the controller is further programmed to actuate the pump system to convey an additional volume of the additive solution into the collection container after the red blood cell collection stage.

Aspect 9. The blood separation device of any one of the preceding Aspects, wherein the controller is further programmed to actuate the pump system to convey blood into the separator at a whole blood inflow rate and to convey plasma separated from the red blood cells in the separator out of the separator at a plasma outflow rate during the red blood cell collection stage, and the separated red blood cells are conveyed from the separator at a rate equal to the difference between the whole blood inflow rate and the plasma outflow rate.

Aspect 10. The blood separation device of any one of the preceding Aspects, wherein the controller is further programmed to actuate the pump system to convey the separated red blood cells through a leukoreduction filter before the separated red blood cells reach the collection container.

Aspect 11. A controller-implemented method of executing a blood separation procedure, comprising: executing a red blood cell collection stage in which blood is conveyed into a separator, red blood cells are separated from the blood in the separator, the separated red blood cells are conveyed from the separator into a collection container, and the red blood cell collection stage is ended when a target volume of blood has been separated in the separator; and determining a percentage of the red blood cells from the target volume of blood that have been collected in the collection container using the equations:

$$\% R B C Recovery = \frac{Total Volume R B C s in R B C Bag}{Total Volume R B C s in WB Bag} \times 100 ,$$

Total Volume RBCs in RBC Bag=V_{RBC Bag}*H_{RBC Bag}, and Total Volume RBCs in WB Bag=Q_{pureRBC-WB}*Procedure Time, wherein Total Volume RBCs in RBC Bag is a volume of red blood cells in the collection container at the end of the blood separation procedure, Total Volume RBCs in WB Bag is the target volume of blood, V_{RBC Bag} is a volume of fluid in the collection container at the end of the blood separation procedure, H_{RBC Bag} is a hematocrit of the fluid in the collection container at the end of the blood separation procedure, Q_{pureRBC-WB} is a volumetric flow rate of pure red blood cells in the blood conveyed into the separator during the red blood cell collection stage, and Procedure Time is the amount of time during the red blood cell collection stage when red blood cells are being separated from the blood in the separator.

Aspect 12. The method of Aspect 11, further comprising establishing steady state separation of the red blood cells from the blood in the separator before conveying the separated red blood cells from the separator into the collection container during the red blood cell collection stage.

Aspect 13. The method of Aspect 12, further comprising using the following equations to determine the percentage of the red blood cells from the target volume of blood that have been collected in the collection container: Q_{pureRBC-WB}=Q_{pureRBC-RBC}, and Q_{pureRBC-RBC}=Q_{RBC}*H_{RBC}, wherein Q_{pureRBC-RBC} is a volumetric flow rate of pure red blood cells in the separated red blood cells conveyed out of the separator during the red blood cell collection stage, Q_{RBC} is a volumetric flow rate of the separated red blood cells conveyed out of the separator during the red blood cell collection stage, and H_{RBC} is a hematocrit of the separated red blood cells conveyed out of the separator during the red blood cell collection stage.

Aspect 14. The method of Aspect 13, further comprising determining H_{RBC} to be equal to a predetermined value based on a separation efficiency of the separator.

Aspect 15. The method of Aspect 13, wherein the separator is configured as a centrifuge, and H_{RBC} is calculated using a centrifugation-based separation efficiency equation.

Aspect 16. The method of Aspect 13, further comprising monitoring the separated red blood cells conveyed out of the separator during the red blood cell collection stage with an optical sensor, wherein the controller is programmed to receive signals from the optical sensor and to determine H_{RBC} based at least in part on said signals.

Aspect 17. The method of any one of Aspects 11-16, further comprising conveying an additive solution into the collection container during the red blood cell collection stage.

Aspect 18. The method of Aspect 17, further comprising conveying an additional volume of the additive solution into the collection container after the red blood cell collection stage.

Aspect 19. The method of any one of Aspects 11-18, wherein said executing the red blood cell collection stage includes conveying blood into the separator at a whole blood inflow rate and conveying plasma separated from the red blood cells in the separator out of the separator at a plasma outflow rate, and the separated red blood cells are conveyed from the separator at a rate equal to the difference between the whole blood inflow rate and the plasma outflow rate.

Aspect 20. The method of any one of Aspects 11-19, wherein the separated red blood cells are conveyed through a leukoreduction filter before reaching the collection container.

It will be understood that the embodiments and examples described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A blood separation device, comprising:
a pump system;
a separator; and
a controller operably connected to the pump system and the separator and programmed to execute blood separation procedure including a red blood cell collection stage in which the controller
actuates the pump system to convey blood into the separator,
actuates the separator to separate red blood cells from the blood in the separator,
actuates the pump system to convey the separated red blood cells from the separator into a collection container, and
ends the red blood cell collection stage when a target volume of blood has been separated in the separator, wherein the controller is further programmed to determine a percentage of the red blood cells from the target volume of blood that have been collected in the collection container using the equations:
$$\% R B C Recovery = \frac{Total Volume R B C s in R B C Bag}{Total Volume R B C s in WB Bag} \times 100 ,$$
$$Total Volume R B C s in R B C Bag = {V}_{RBC Bag} * {H}_{RBC Bag} , and$$
$$Total Volume R B C s in WB Bag = {Q}_{pureRBC - WB} * Procedure Time ,$$
wherein
Total Volume RBCs in RBC Bag is a volume of red blood cells in the collection container at the end of the blood separation procedure,
Total Volume RBCs in WB Bag is the target volume of blood,
V_{RBC Bag} is a volume of fluid in the collection container at the end of the blood separation procedure,
H_{RBC} Bag is a hematocrit of the fluid in the collection container at the end of the blood separation procedure,
Q_{pureRBC-WB} is a volumetric flow rate of pure red blood cells in the blood conveyed into the separator during the red blood cell collection stage, and
Procedure Time is the amount of time during the red blood cell collection stage when red blood cells are being separated from the blood in the separator.

2. The blood separation device of claim 1, wherein the controller is further programmed to establish steady state separation of the red blood cells from the blood in the separator before actuating the pump system to convey the separated red blood cells from the separator into the collection container during the red blood cell collection stage.

3. The blood separation device of claim 2, wherein the controller is further programmed to use the following equations to determine the percentage of the red blood cells from the target volume of blood that have been collected in the collection container:
$${Q}_{pureRBC - WB} = {Q}_{pureRBC - RBC} , and$$
$${Q}_{pureRBC - RBC} = {Q}_{RBC} * {H}_{RBC} ,$$
wherein
Q_{pureRBC-RBC} is a volumetric flow rate of pure red blood cells in the separated red blood cells conveyed out of the separator during the red blood cell collection stage,
Q_{RBC} is a volumetric flow rate of the separated red blood cells conveyed out of the separator during the red blood cell collection stage, and
H_{RBC} is a hematocrit of the separated red blood cells conveyed out of the separator during the red blood cell collection stage.

4. The blood separation device of claim 3, wherein the controller is further programmed to determine H_{RBC} to be equal to a predetermined value based on a separation efficiency of the separator.

5. The blood separation device of claim 3, wherein
the separator is configured as a centrifuge, and
the controller is further programmed to calculate H_{RBC} using a centrifugation-based separation efficiency equation.

6. The blood separation device of claim 3, further comprising an optical sensor configured to monitor the separated red blood cells conveyed out of the separator during the red blood cell collection stage, wherein the controller is programmed to receive signals from the optical sensor and to determine H_{RBC} based at least in part on said signals.

7. The blood separation device of any one of the preceding claims, wherein the controller is further programmed to actuate the pump system to convey an additive solution into the collection container during the red blood cell collection stage.

8. The blood separation device of claim 7, wherein the controller is further programmed to actuate the pump system to convey an additional volume of the additive solution into the collection container after the red blood cell collection stage.

9. The blood separation device of any one of the preceding claims, wherein
the controller is further programmed to actuate the pump system to convey blood into the separator at a whole blood inflow rate and to convey plasma separated from the red blood cells in the separator out of the separator at a plasma outflow rate during the red blood cell collection stage, and
the separated red blood cells are conveyed from the separator at a rate equal to the difference between the whole blood inflow rate and the plasma outflow rate.

10. The blood separation device of any one of the preceding claims, wherein the controller is further programmed to actuate the pump system to convey the separated red blood cells through a leukoreduction filter before the separated red blood cells reach the collection container.

11. A controller-implemented method of executing a blood separation procedure, comprising:
executing a red blood cell collection stage in which
blood is conveyed into a separator,
red blood cells are separated from the blood in the separator,
the separated red blood cells are conveyed from the separator into a collection container, and
the red blood cell collection stage is ended when a target volume of blood has been separated in the separator; and
determining a percentage of the red blood cells from the target volume of blood that have been collected in the collection container using the equations:
$$\% R B C Recovery = \frac{Total Volume R B C s in R B C Bag}{Total Volume R B C s in WB Bag} \times 100 ,$$
$$Total Volume R B C s in R B C Bag = {V}_{RBC Bag} * {H}_{RBC Bag} , and$$
$$Total Volume R B C s in WB Bag = {Q}_{pureRBC - WB} * Procedure Time ,$$
wherein
Total Volume RBCs in RBC Bag is a volume of red blood cells in the collection container at the end of the blood separation procedure,
Total Volume RBCs in WB Bag is the target volume of blood,
V_{RBC Bag} is a volume of fluid in the collection container at the end of the blood separation procedure,
H_{RBC Bag} is a hematocrit of the fluid in the collection container at the end of the blood separation procedure,
Q_{pureRBC-WB} is a volumetric flow rate of pure red blood cells in the blood conveyed into the separator during the red blood cell collection stage, and
Procedure Time is the amount of time during the red blood cell collection stage when red blood cells are being separated from the blood in the separator.

12. The method of claim 11, further comprising establishing steady state separation of the red blood cells from the blood in the separator before conveying the separated red blood cells from the separator into the collection container during the red blood cell collection stage.

13. The method of claim 12, further comprising using the following equations to determine the percentage of the red blood cells from the target volume of blood that have been collected in the collection container:
$${Q}_{pureRBC - WB} = {Q}_{pureRBC - RBC} , and$$
$${Q}_{pureRBC - RBC} = {Q}_{RBC} * {H}_{RBC} ,$$
wherein
Q_{pureRBC-RBC} is a volumetric flow rate of pure red blood cells in the separated red blood cells conveyed out of the separator during the red blood cell collection stage,
Q_{RBC} is a volumetric flow rate of the separated red blood cells conveyed out of the separator during the red blood cell collection stage, and
H_{RBC} is a hematocrit of the separated red blood cells conveyed out of the separator during the red blood cell collection stage.

14. The method of claim 13, further comprising determining H_{RBC} to be equal to a predetermined value based on a separation efficiency of the separator.

15. The method of claim 13, wherein
the separator is configured as a centrifuge, and
H_{RBC} is calculated using a centrifugation-based separation efficiency equation.

16. The method of claim 13, further comprising monitoring the separated red blood cells conveyed out of the separator during the red blood cell collection stage with an optical sensor, wherein the controller is programmed to receive signals from the optical sensor and to determine H_{RBC} based at least in part on said signals.

17. The method of any one of claims 11-16, further comprising conveying an additive solution into the collection container during the red blood cell collection stage.

18. The method of claim 17, further comprising conveying an additional volume of the additive solution into the collection container after the red blood cell collection stage.

19. The method of any one of claims 11-18, wherein
said executing the red blood cell collection stage includes conveying blood into the separator at a whole blood inflow rate and conveying plasma separated from the red blood cells in the separator out of the separator at a plasma outflow rate, and
the separated red blood cells are conveyed from the separator at a rate equal to the difference between the whole blood inflow rate and the plasma outflow rate.

20. The method of any one of claims 11-19, wherein the separated red blood cells are conveyed through a leukoreduction filter before reaching the collection container.
